(19) United States
(12) Patent Application Publication
(10) Pub. No.: US 2014/0056495 A1
(43) Pub. Date: Feb. 27, 2014

(54) IMAGING CALIBRATION DEVICE

(71) Applicant: Michel Janssens, Grez-Doiceau (BE)

(72) Inventor: Michel Janssens, Grez-Doiceau (BE)

(73) Assignee: Materialise N.V., Leuven (BE)

(21) Appl. No.: 14/113,430

(22) PCT Filed: May 4, 2012
(86) PCT No.: PCT/EP12/58242

§ 371 Date: Oct. 23, 2013

Related U.S. Application Data
(60) Provisional application No. 61/482,276, filed on May 4, 2011.

(30) Foreign Application Priority Data
May 4, 2011 (GB) 1107385.5

(51) Int. Cl. G06T 5/00; A61B 6/00

(52) U.S. Cl. CPC G06T 5/006; A61B 6/582
USPC 382/128; 378/207

(57) ABSTRACT

The present invention relates to calibration devices, the use thereof and methods for the in-line geometric and grey-value correction of data obtained from imaging

N devices such as x-ray radiography. The calibration devices according to the invention comprise a set of two or more interconnected calibration components and a means for mounting the set of calibration components on the body.

### FIELD OF THE INVENTION

The present invention relates to calibration devices, the use thereof and methods for the in-line geometric and grey-value correction of data obtained from imagining devices such as x-ray radiography, ultrasonography, MRI or CT.

### BACKGROUND

Traditionally, medical imaging techniques such as x-ray radiography are mainly used for qualitative measurements and diagnostics, e.g. to see if a bone is broken or not, or to see if a tumor is present or not. However, the modern applications of x-ray imaging for quantitative measurements have given rise to new requirements concerning the accuracy of x-ray imaging.

First of all, the geometrical accuracy is a common issue in dimensional metrology. If the geometric accuracy of an image is excellent, then the distance between two features on an object can be unambiguously derived from the distance between those features on the image of that object, for example by multiplying the distances on the image by a specific scaling factor. However, due to various mechanical and optical effects, radiographic images such as x-ray images can be distorted. Known effects are the pincushion effect and magnification or minification of various body parts relative to their distance from the imaging surface. The result is that a certain distance on the image does not give an accurate estimate of the real distance, e.g. dimensions of certain anatomical parts. Thus, uncalibrated x-ray images only offer qualitative information, not quantitative information.

A second issue is the grey-value accuracy of radiographic images. The grey-value can vary between different measurements, and even within a single measurement, for example due to inhomogeneity of the radiation source or the unstable sensitivity of the radiographic plate or detector.

As a result of the limited accuracy, it is for example hard to make a distinction between a five percent increase or decrease in size of a tumor, when comparing two x-ray images of the same body part taken at different times. On the other hand, when measuring osteoporosis, one can find a bone density based on the grey-values in a certain area of the image, but it is impossible to compare the grey-levels in this image to the levels in an image taken at a later time. In principle, this problem could be solved by consistently using calibrated x-ray machines. In practice, this is so cumbersome that it is rarely done.

In the past, solutions have been proposed for some of these problems.

US patent application 2008/273665 (Rolle, Boots) describes a device and method to accurately measure the anatomy of a patient undergoing a radiographic procedure, utilizing a sphere of known dimensions attached to a flexible member. The sphere is manipulated into the correct position and remains stationary during the procedure. U.S. Pat. No. 6,459,772 (Wiedenhoefer et al.) describes a radiographic reference marker, containing a sphere of a known size, contained in a radiolucent housing. The sphere is attached to a patient in the same plane as the anatomical target of the X-ray. Although these inventions can be used for determination of the magnification or minification of an image, they do not allow a more rigorous correction of the geometric distortions in the image, and the high radiopacity of the markers does not allow a grey-value correction. Moreover, although these devices offer a high degree of freedom for positioning the marker relative to the body, they do not provide a means to do this in a reproducible way. Additionally, these techniques only provide reference markers in one area of the image.

U.S. Pat. No. 5,951,475 (Gueziec et al.) describes methods and a system for geometrically calibrating x-ray projection images using a calibration device that includes radio-opaque markers, wherein the calibration device can be manipulated by a robot. However, this system requires special and expensive equipment, and further requires obtaining a plurality of images of the region of interest. Moreover, the system only allows geometrical calibration, not grey-value correction.

Accordingly, there is a need for improved methods and tools for calibration of images.

### SUMMARY OF THE INVENTION

The present invention relates to imaging calibration devices and methods for the calibration of data obtained by imaging techniques. More particularly methods and tools involving (in-line) geometric and/or grey-value correction of data obtained by imaging techniques such as radiography, ultrasound, x-ray computed tomography (CT) and magnetic resonance imaging (MRI).

According to one aspect methods for correction and/or calibration of an image of an object are provided which make use of calibration components having specified radiopacity and geometrical position. In particular embodiments, the methods may comprise the steps of:

I) providing an image wherein said image includes an image of one or more sets of two or more non-identical and interconnected calibration components; and

II) correcting the image based on the information obtained from the measurement of the image of the calibration components.

More particularly the image includes the image of the object and an image of one or more sets of two or more non-identical and interconnected calibration components and the image of the object is corrected based on the information obtained from the measurement of the image of the calibration components.

Typically, the two or more non-identical and interconnected or calibration components differ at least in radiopacity.

In particular embodiments, at least one of the sets of calibration components comprises three or more non-identical calibration components, of which two or more differ at least in radiopacity and the three calibration components are interconnected to form a fixed three-dimensional geometrical figure.

In particular embodiments, step I) may involve providing an image wherein the image includes an image of one or more sets of calibration components as described herein. In certain embodiments, the methods provided are methods for correction and/or calibration of an image of at least part of a region of interest of a patient and step I) comprises the steps of:

a. application of a medical imaging calibration device as described herein to the patient in the region of interest; and

b. acquisition of an image of the region of interest including the medical imaging calibration device.

In particular embodiments, methods for correction of images as provided herein comprise a grey-value correction of the image. In further embodiments, such methods may comprise the steps of:
identification of two or more calibration components in the image;
determination of the grey value of the identified calibration components in the image;
calculating the grey value error based on the difference between the determined grey value and the calculated grey value; and
applying the correction on the image.

In particular embodiments, the methods may involve the use of imaging calibration devices comprising one or more sets of two or more non-identical and interconnected or interconnectable calibration components wherein the non-identical and interconnected or interconnectable calibration components differ in radiopacity.

In particular embodiments, the method for correction of images according to the present invention comprises a geometrical correction of the image. In further embodiments, the methods may comprise the steps of:
identification of at least three calibration components in the image;
determination of the 3D position of each calibration component;
calculating of the geometrical error on the image; and
applying the correction on the image.

In particular embodiments, methods for correcting and/or calibrating an image are provided, which methods comprise

I) providing an image wherein said image includes an image of one or more sets of two or more non-identical and interconnected or interconnectable calibration components ; and

II) correcting the image based on the information obtained from the measurement of the geometry and grey values of said image of said calibration components.

In particular embodiments, methods for correction of images are provided which are methods for correction of images of (at least part of) a patient and which may comprise providing information on the position and the shape of the (relevant part of the) patient.

In particular embodiments, the methods for correction and/or calibration of images provided herein are further based on the determination of errors in more than one area in the image.

In certain embodiments, the methods for correction of images provided may further comprise the step of removing the images of the calibration components from the image. In particular embodiments, the correction is an in-line correction.

In a further aspect, calibration devices are provided comprising a set of two or more non-identical and interconnected or interconnectable calibration components, wherein at least two calibration components have a radiopacity between, and not including, 0 and 1. More particularly, two or more non-identical and interconnected or interconnectable calibration components of the device have at least a different radiopacity. In further particular embodiments, the set comprises three or more non-identical calibration components and further comprises means for interconnecting said calibration components to form a fixed three-dimensional geometrical figure.

The set of calibration components may further comprise a means, more particularly a separate structure for mounting and/or positioning the set of calibration components on the body. In particular embodiments, the interconnected calibration components have fixed relative positions.

The calibration devices as claimed herein allow for a geometrical and/or grey-value correction of images such as medical images. More particularly, they allow for a combination of geometrical and grey-value correction of images. In particular embodiments, the devices of the present invention allow for the correction of local inhomogeneities in an object. This significantly enhances the quality of the image, which is of particular interest in the context of medical images. Thus, in particular embodiments, the medical imaging calibration devices claimed herein are of use for increasing the quality of medical images.

In particular embodiments, the calibration devices claimed herein can be used for grey-value correction of images such as medical images. In particular embodiments, at least two calibration components in the set have a different radiopacity. In certain embodiments, the calibration components are spherical.

In particular embodiments, the centers or longitudinal axis of said interconnected calibration components (i.e. when interconnected) respectively form the vertices or the edges of an imaginary polyhedron. In further embodiments, the centers of the interconnected calibration components form the vertices of an imaginary polyhedron. In certain embodiments, the interconnected calibration components form a tetrahedron. In particular embodiments, the set of interconnected calibration components forms a polyhedron with at least one calibration component placed inside the polyhedron. In further embodiments, the set of interconnected calibration components forms a polyhedron with at least one calibration component placed in the center of the polyhedron.

In particular embodiments, at least one of the calibration components is hollow. In further embodiments, the ratio of the outer and inner diameter of at least one calibration component is different from the ratio of the outer and inner diameter of another calibration component.

In certain embodiments, the outer diameter of at least one calibration components is different from the outer diameter of another calibration component.

Advantageously, the calibration devices claimed herein may be used for (independently) correcting different areas in one image. This way, local inhomogeneities in one area of the image do not influence the correction in another part of the image. To ensure this, in particular embodiments, the calibration devices according to the present invention comprise at least two sets of calibration components.

In particular embodiments, the calibration components are made of a polymer containing metal, metal oxide or metal sulfate particles.

In particular embodiments, the claimed calibration devices further comprise a housing which covers at least part of the calibration components. In particular embodiments, the housing may comprise a feature which facilitates positioning of the device on the object of interest, such as a human body part. In particular embodiments, the housing comprises an (patient-specific) engagement surface.

In certain embodiments, the means for mounting the set(s) of calibration components is a garment.

In particular embodiments, the calibration components in the calibration devices according to the present invention are adapted to a certain tissue type or a certain region of interest of the body.

In certain embodiments, the calibration device according to the present comprises a set of at least five non-identical spheres which have an x-ray opacity between and not including 0 and 1, wherein the spheres are interconnected or interconnectable in such way that the centers of at least four of the spheres form the vertices of an imaginary polyhedron, and one sphere is located inside the polyhedron.

A further aspect of the use of the medical imaging calibration components as described herein is provided, for quantitative measurement on data obtained from x-rays, ultrasound, CT and MRI.

### BRIEF DESCRIPTION OF THE DRAWINGS

The following description of the figures of specific embodiments of the invention is merely exemplary in nature and is not intended to limit the present teachings, their application or uses. Throughout the drawings, corresponding reference numerals indicate like or corresponding parts and features.

FIG. 1 Illustration of radiographic imaging, showing a radiation source (7), an x-ray radiation beam (8), a region of interest (9) and a detector (10).

FIG. 2 Medical imaging calibration device (1) according to a particular embodiment of the present invention, comprising several sets (2) of calibration components, and a means (3) for mounting the set of calibration components on the body.

FIG. 3 Set (2) of interconnected calibration components (4, 5) according to a particular embodiment of the present invention, comprising four outer calibration components (4), an inner calibration component (5) and connections (6) between the inner calibration component (5) and the outer calibration components (4). The center points of the four outer calibration components (4) form the vertices of an imaginary polyhedron, i.e. a tetrahedron. The inner calibration component (5) is located in the centre of the tetrahedron.

FIG. 4 Set (2) of interconnected calibration components (4, 5) according to a particular embodiment of the present invention, comprising five outer calibration components (4), an inner calibration component (5) and connections (6) between the inner calibration component (5) and the outer calibration components (4). The center points of the five outer calibration components (4) form the vertices of an imaginary polyhedron, i.e. a (square) pyramid. The inner calibration component (5) is located in the centre of the pyramid.

FIG. 5 Flow diagram of exemplary steps in methods according to particular embodiments of the invention for in-line correction of radiographic images.

FIG. 6 A-G: Set (2) of 4 interconnected calibration components (4, 5) according to a particular embodiment of the present invention provided in a housing (11). FIG. 7 A-D: Set (2) of 4 interconnected calibration components (4, 5) according to a particular embodiment of the present invention provided in a housing (11).

FIG. 8 A-D: Set (2) of 4 interconnected calibration components (4, 5) according to a particular embodiment of the present invention provided in a housing (11).

FIG. 9 A, B: Configuration of various sets (2) of interconnected calibration components (4, 5) according to a particular embodiment of the present invention.

FIG. 10 A-C: Set (2) of interconnected calibration components (4, 5) according to a particular embodiment of the present invention provided in a housing (11).

In the figures, the following numbering is used:

1—calibration device; 2—set of calibration components; 3—means for mounting; 4, 5—calibration component; 6—connection; 7—radiation source; 8—radiation beam; 9—region of interest; 10—detector; 11—housing; 12—engagement surface; 13—lid.

### DETAILED DESCRIPTION

The present invention will be described with respect to particular embodiments but the invention is not limited thereto but only by the claims. Any reference signs in the claims shall not be construed as limiting the scope thereof.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments of the invention described herein are capable of operation in other sequences than described or illustrated herein.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

The recitation of numerical ranges by endpoints includes all numbers and fractions subsumed within the respective ranges, as well as the recited endpoints.

All documents cited in the present specification are hereby incorporated by reference in their entirety.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. The terms or definitions used herein are provided solely to aid in the understanding of the invention.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the claims, any of the claimed embodiments can be used in any combination.

The term “grey-value” as used herein refers to any measure for intensity or intensity variation in an image. This intensity can be represented as a certain greyscale going from white to black, or a certain color tint, e.g. as in false-color images. Intensities may also be represented as a certain number.

The term “set” as used herein when referring to a set of calibration components, includes two or more interconnected or interconnectable components.

The term “opacity” as used herein refers to the measure of impenetrability of an object or material to a specific kind of radiation. The term “radiopacity” while in principle referring specifically to X-rays and similar radiation, is also generally used herein interchangeably with “opacity”. Opacity depends on the frequency of the light being considered. Opacity can be quantified in different ways in including the mass attenuation coefficient or the absorption rate.

The term “specific absorption rate” as used herein refers to a measure of the rate at which energy is absorbed by an object or anatomical part when exposed to electromagnetic radiation, sound, particles or other energy or matter. It is defined as the power absorbed per mass and has units of watts per kilogram. The value of the specific absorption rate varies depending on the frequency of the electromagnetic radiation or sound, or on the type of particles or other energy or matter. Whenever a specific absorption rate is mentioned in this text, the corresponding electromagnetic radiation, sound, particles or other energy or matter is the type of radiation, sound, particles or other energy or matter used in a medical imaging technique. For example, in x-ray radiography, the electromagnetic radiation is x-radiation in a certain frequency range.

The term “attenuation coefficient” as used herein refers to a quantity that characterizes how easily a material or medium can be penetrated by a beam of light, sound, particles, or other energy or matter. A large attenuation coefficient means that the beam is quickly “attenuated” (weakened) as it passes through the medium, and a small attenuation coefficient means that the medium is relatively transparent to the beam. Attenuation coefficient is measured using units of reciprocal length. Whenever an attenuation coefficient is mentioned in this text, the corresponding electromagnetic radiation, sound, particles or other energy or matter is the type of radiation, sound, particles or other energy or matter used in an imaging technique. For example, in x-ray radiography, the electromagnetic radiation is x-radiation in a certain frequency range.

Imaging techniques envisaged for the application of the present invention include techniques such as radiography, ultrasound, x-ray computed tomography (CT), thermography, magnetic resonance imaging (MRI) and nuclear medicine such as positron emission tomography (PET).

The term “medical imaging” as used herein refers to techniques and processes used to create images of the human or animal body (or parts and function thereof), typically for clinical purposes (medical procedures seeking to reveal, diagnose or examine disease) or medical science (including the study of normal anatomy and physiology).

The term “(medical) image” as used herein refers to an image obtained by a (medical) imaging technique. The term “image” further includes images obtained by radiography, ultrasound, CT, thermography, MRI and nuclear medicine, also when used for non-medical purposes.

In one aspect, imaging calibration devices are provided. More particularly, calibration devices are provided which make it possible to obtain an improved geometrical accuracy and grey-value accuracy of x-ray, ultrasound, x-ray computed tomography (CT) and/or magnetic resonance imaging (MRI) images, more particularly of a region of interest of a body. In particular embodiments, the imaging calibration devices provided are radiographic calibration devices.

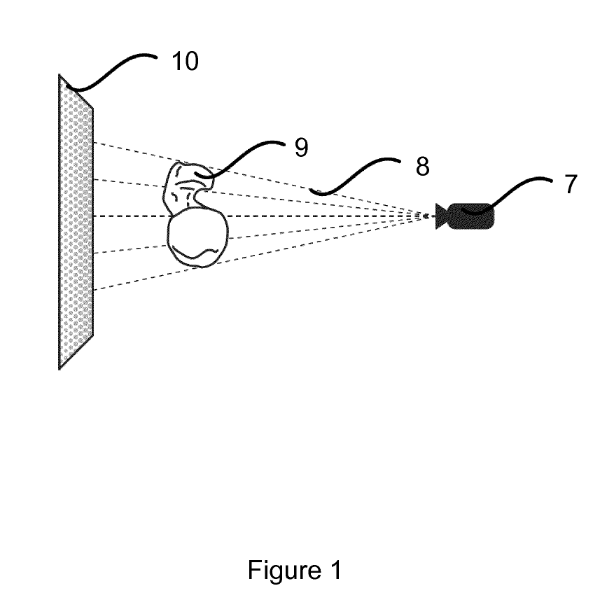

A schematic illustration of an exemplary radiographic imaging is shown in FIG. 1. A (x-ray) radiation source (7) produces a (x-ray) radiation beam (8). A region of interest (9), for example a body part, is located between the radiation source and a detector (10). As the beam is divergent, the image of the area of interest on the detector is distorted. For example, the areas of the region of interest near the edge of the beam are magnified more than the areas in the center of the beam. Additionally, areas of the region of interest closer to the detector are magnified less than areas further away from the detector. Instability and inhomogeneity of the beam source may cause further distortions in the image. Therefore, for quantitative measurements, an image needs to be calibrated. Preferably, the image is calibrated based on measurement of the errors (distortions) in different areas of the image.

The calibration devices claimed herein contain a set of two or more, more particularly three or more non-identical interconnectable or interconnected calibration components. More particularly, the calibration devices envisaged herein comprise at least one set of two or more non-identical interconnected or interconnectable calibration components, wherein two or more non-identical interconnected or interconnectable calibration components have a different radiopacity. More particularly, the radiopacity of the calibration components is between, and not including, 0 and 1. The difference in radiopacity can be ensured in different ways, such as, but not limited to optical density of the material, size etc. as will be detailed below. The use of at least two calibration components with different radiopacity further improves the grey value correction.

It is envisaged that during use of the calibration devices, the position of the calibration components is fixed relative to each other. Accordingly, where the calibration components are provided as individual interconnectable components, the set further comprises means or features for interconnecting the calibration components to form a fixed three-dimensional geometrical figure.

Different implementations of interconnecting calibration components are envisaged. Thus, each calibration component in the set may be either directly or indirectly connected to the other calibration components in the set. For example, each calibration component can be linked directly to each other calibration component in the set. In particular embodiments, some calibration components in the set are only linked to one other calibration component in that set. Thus, some of the calibration components in the set may be connected or connectable indirectly. For example, in a set of three calibration components the first component can be connected directly to the second component and the third component can also be connected directly to the second component, but not directly to the first component. In this embodiment, there is an indirect connection between the first and third component. In this way, the number of connections is minimized, so also the amount of artifacts in the medical image due to the connections is reduced. Alternatively, all of the components can be directly connected or connectable to each other.

As indicated above, the calibration devices envisaged herein may comprise one or more calibration components. The number of components will to a certain extent depend on the applications of the calibration device. A set comprising 1 or 2 calibration components allows for the calibration of the grey levels in an image and allows for the calculation of the degree of magnification or minification in the image (2-dimensional (2D) correction). For a 3D geometrical correction, at least 3 calibration components are required. Therefore, in particular embodiments, the set of interconnected calibration components comprises at least two, more particularly at least three calibration components. The presence of four calibration components in a device according to the present invention allows for a grey-value and geometrical correction, and allows to assess the calibration of the medical imaging device. Thus, in particular embodiments, the set of interconnected calibration components comprises at least four calibration components. A set of five calibration components allows for a full spatial, 3D and grey-value correction, without the need of further information about the calibration of the medical imaging device. Therefore, in particular embodiments, the set of interconnected calibration components comprises at least five calibration components. In addition to the above, it is noted that the accuracy of correction of spatial artifacts is further increased with the number of calibration components provided at different positions in the image. Thus, it is further envisaged that in particular embodiments sets of 6, 7, 8, 9, 10 or more calibration components are provided in the devices according to the present invention.

Within each set, the calibration components are provided such that they can be set in one or more fixed positions relative to each other. In particular embodiments, when interconnected, the calibration components do not touch each other. Practically, overlap of the calibration components in the image should be minimized and if possible avoided. It is preferred but not required (as illustrated below) that the distance between the calibration components, or at least parts thereof, is at least 10 pixels on the medical image. Moreover, the size of the calibration components is preferably such that the size of each calibration component is several pixels in the image, preferably 10 or more pixels. Typically, the accuracy of the geometric and/or grey-value calibration increases if the calibration components spread out over more pixels in the image.

The nature of the connection between the calibration components is not critical. For example, the calibration components may be connected by connecting elements such as, but not limited to a straight or bent pin or rod. Additionally or alternatively, the connection of two or more of the calibration components in a set may be obtained via direct contact between the calibration components. Direct contact between the calibration components is particularly suitable when at least one of the calibration components is rod-shaped.

In particular embodiments, the sets of calibration devices are provided as irreversibly fixed interconnected structures, whereby interconnection can be ensured in any way such as gluing or manufacturing in one piece. In further embodiments, the calibration components are reversibly interconnectable and the set may comprise means or features to allow connection of the calibration components so as to form a fixed two-dimensional and more particularly three-dimensional structure. In particular embodiments, it is preferred that the connection upon use of the device is stable and strong and does not interfere with the imaging of the calibration components. In particular embodiments, the connection between the calibration components in the set is ensured by one or more dedicated connecting features and/or connecting elements. In further particular embodiments, the connection can ensure a fixed relative position of the calibration components. More particularly, the nature of the connecting feature(s) and/or connecting elements ensures that the calibration component can be positioned at a distance from one or more other calibration components which is at least equal to the smallest diameter of the connecting element.

The connecting features may be incorporated in the calibration components themselves. For instance the connecting element can be, e.g. a threaded cylinder, which fits into a threaded opening (as connecting feature) present on one or more calibration components. Additionally or alternatively, the connecting feature(s) may be external components (e.g. a housing ensuring that the different components are placed in a fixed position relative to each other). A set may also comprise a combination of rigidly interconnected and interconnectable calibration components (e.g. to reduce packaging size). In any case, it is important that the calibration components are or can be rigidly fixed in a specified position relative to each prior to use, such that the relative position of the components does not change, e.g. upon fixation of the set on a body part.

In particular embodiments, the dedicated connecting features and/or connecting elements ensure that one or more of the calibration components in the set can be connected in different ways to the other calibration components, in such way that the relative position of the calibration components is adjustable but can thereafter be securely fixed prior to use.

Thus, the calibration components in the set have fixed relative positions, or can be interconnected to take on fixed relative positions. In particular embodiments, the set of interconnected calibration components is provided as a fixed set of calibration components. More particularly, the calibration device is provided and optionally fabricated as a single piece.

The calibration devices as envisaged herein may optionally comprise a means for mounting or positioning the one or more sets of calibration components on a body or body part. While “positioning” in this context implies emphasis on the correct placement of the sets of calibration components, “mounting” in this context refers more to the ability to maintain a fixed position on an object of interest such as a body part. More particularly the means for mounting or positioning the set of calibration components on a body or body part is a structure which is not part of the calibration components as such, but can be (removably) attached thereto. In particular embodiments, the means for mounting or positioning facilitate the positioning or mounting of the set of calibration components on the body or on a body part, in such a way that the calibration components are near to a certain region of interest on the body, optionally in a predefined position. In particular embodiments, the feature is such that it allows for mounting a plurality of calibration components near the region of interest. In particular embodiments, the feature for positioning and/or mounting further ensures that the relative position of the set of calibration components to the region of interest does not vary during the image acquisition. The region of interest may be a region of a bone, of tissue, a region known or expected to comprise a tumor, etc. Examples of features envisaged for positioning and/or mounting are detailed herein below.

Additionally or alternatively, the relative position of two or more calibration components in a set may be secured by a housing. Such a housing is typically a structure which protects at least one of the calibration component and/or secures at least two or more of the calibration components relative to each other. Such a housing may further help to protect the shape and integrity of the calibration components, for example when these are made from brittle materials. In these embodiments, the housing preferably covers at least part of the calibration component(s). In particular embodiments, the housing covers at least 30% of one or more of the calibration components. In particular embodiments, the housing covers at least 30% of the calibration component(s) on the surface of the calibration components which form(s) the exterior surface of the set of calibration components. In particular embodiments, the housing consists of a single part, and may be connected to one or more of the (set of) calibration components via a snap-fit or clip-on mechanism. In other embodiments, the housing may comprise two or more removably connectable parts, for facilitating insertion and removal of the set of calibration components into the housing. In particular embodiments, the two or more parts of the housing are removably connectable via a snap-fit system.

Typically, the housing is made from a material with a negligible attenuation coefficient and/or radiopacity, such that it does not interfere with the image of the calibration components. Suitable materials include but are not limited to polymers such as polystyrene, polyvinylchloride, polyesters, polypropylene, polycarbonate, poly(methyl methacrylate), polyethylenteterephthalate, polyamides or blends thereof. These materials have a low radiopacity, while still providing the necessary strength for securing the position of the calibration components. In particular embodiments, each set of calibration components of the calibration device described herein is provided with a separate housing. In other embodiments, two or more sets of calibration components of the calibration device may be comprised in the same housing.

The housing may further be suitable as a means for positioning and/or mounting the set of calibration components on an object of interest such as on the body. Thus, in particular embodiments, the means for positioning and/or mounting is integrated in a housing. However, the devices may also comprise dedicated housing and positioning or mounting features. This will be explained further in this text.

In certain embodiments, the calibration components in the set are connected by being embedded in a radiolucent material, i.e. a material with a radiopacity of 0, or close to 0.

The shape and size of the calibration components of the calibration devices according to the present invention is not critical. In particular embodiments, the calibration components are spheres, hemispheres, ellipsoids, cubes, tetrahedrons, pyramids, rods, discs, washers, wires, or any combination thereof.

In a particular embodiment, some of the calibration components are spheres. In certain embodiments, all the calibration components are spherical, or substantially spherical. Spheres are metrologically very stable to measure, easily detected in an image by image processing software, and easy to produce accurately.

In particular embodiments, one or more of the calibration components are rods. Typically, the rods are cylindrical and have an aspect ratio (i.e. length divided by width) ranging between 2 and 10. In certain embodiments, all the calibration components are rods.

In certain embodiments, the calibration components comprise a combination of one or more spheres and one or more rods. Besides acting as calibration components, the rods may further act as connecting elements for connecting the spheres. In particular embodiments, one or more calibration components comprise a rod which is connected to a sphere.

Each calibration component in a set may be identified in an image via unique features of that calibration component, or via its relative position with respect to an identifiable calibration component. Accordingly, most or all of the calibration components in a set are non-identical, to ensure an unambiguous identification of the calibration components in an image. The calibration components in a set may therefore have a different shape, size, inner diameter, outer diameter, mark, identification code, radiopacity, or a combination thereof. These features will be explained in more detail herein below.

The identification of calibration components can be facilitated and improved if the calibration components are hollow. Thus, in particular embodiments, one or more of the calibration components in the set are hollow. In further particular embodiments, one calibration component in the set is solid, and one or more, more particularly all other calibration components in the set are hollow. In alternative particular embodiments, all of the calibration components in the set are hollow. More particularly, calibration devices are provided wherein one or more of the calibration components in the set are hollow spheres. In particularly envisaged embodiments, one calibration component in the set is a solid sphere, and all other calibration components in the set are hollow spheres. In alternative particular embodiments, all the calibration components in the set are hollow spheres. The ratio between the large and small radius, i.e. outer and inner radius, of a hollow sphere is invariant under projection, which facilitates identification of the calibration components in an image. In other embodiments, none of the calibration components are hollow.

The identification of the calibration components in an image is also facilitated if the calibration components in the set have different (outer) diameters or sizes. Thus, In particular embodiments, the calibration components of one set have a different outer diameter or size. If the calibration device described herein comprises two or more rod-shaped calibration components, the rods may have a different width and/or length. In certain embodiments, the calibration device comprises two or more rod-shaped calibration components, all having different widths.

In particular embodiments of the calibration devices of the invention, the (maximal) outer diameter of at least one calibration component is different from the outer diameter of another calibration component in the set.

It is noted that where calibration devices comprising hollow calibration components are envisaged, the ratio between outer and inner diameter may vary. In particular embodiments, the ratio of the outer and inner diameter of at least one of the hollow calibration components is different from the ratio of the outer and inner diameter of another calibration component. In particular embodiments, each calibration component in the set of interconnected calibration components has a ratio of the outer and inner diameter which is different from the ratio of the outer and inner diameter of all other calibration components in the set. If each calibration component has a unique ratio of the outer and inner diameter, this facilitates identification of the different calibration components in an image. In more particular embodiments the devices of the present invention, he calibration components are hollow spheres, and the ratio between the inner and outer diameter for each sphere is different.

For example, the inner diameter of the calibration components which are hollow spheres or cylindrical rods is typically between 9.9 and 0.1 cm, preferably between 2.9 and 0.2 cm, more preferably between 0.9 and 0.2 cm and even more preferably between 0.6 and 0.3 cm. The outer diameter of the calibration components which are hollow spheres or cylindrical rods is typically between 10 and 0.4 cm, preferably between 3 and 0.4 cm, more preferably between 1 and 0.4 cm, and even more preferably between 0.7 and 0.5 cm, provided that the outer diameter is larger than the inner diameter. The diameter of the calibration components which are solid spheres is typically between 10 and 0.1 cm, preferably between 3 and 0.3 cm, more preferably between 1 and 0.3 cm and even more preferably between 0.7 and 0.3 cm.

In particular embodiments, the calibration components can additionally or alternatively be identified in a different way, e.g. by marking of the calibration component. The calibration components may be provided with an identification tag or code which allows identification thereof in the image. Suitable methods for marking an object such that the marking is visible on the image taken, depend on the nature of the imaging device and are known to the skilled person. Additionally or alternatively, the calibration components can have a different size and/or shape.

The envisaged use of the calibration devices and methods of the present invention is in particular embodiments based on the provision of reference values for grey values of the image. Indeed, in particular embodiments, the calibration devices of the present invention are used to calibrate grey values in medical imaging. For this purpose, at least two of the calibration components must ensure absorption of some, but not all of the radiation used in the medical imaging. This implies that at least two of the calibration components present in a set of the device according to the present invention should have a radiopacity between, and not including, 0 and 1. A material or object with radiopacity 0 allows total transmission of the radiation used for the medical imaging. A Material or object with radiopacity 1 totally blocks the radiation used for the medical imaging from the radiation source to the film or detector. If a calibration component has a radiopacity between 0 and 1, this means that the image of the calibration component will comprise grey-values between the minimum and maximum grey-value. As radiopacity is dependent on the frequency used, the radiopacity of the calibration components is determined by their envisaged use. Thus, where the purpose is calibration of X-ray images, the radiopacity of the components should be between 0 and 1 for X-rays, etc. It can however be envisaged that a device is provided, comprising at least one set of components, wherein at least two of the calibration components present in the set have an opacity of between 0 and 1 for different imaging methods envisaged.

Thus, in preferred embodiments, at least two of the calibration components of the calibration devices of the present invention have a radiopacity between 0 and 1. More particularly, these calibration components are made from a material with a radiopacity between, and not including, 0 and 1. In more particular embodiments, all of the calibration components in the set have a radiopacity between 0 and 1.

It is further noted that the quality of the correction will improved when the opacity of the calibration components is in the range of the opacity values of the region of interest. Thus, in further particular embodiments, the radiopacity or the specific absorption rate (SAR) of each calibration component has a predetermined value, more specifically a value which is selected based on the opacity of the region of interest. Moreover, to ensure optimal correction of the grey values, the provision of calibration components with different opacities is also of interest. Thus in particular embodiments, the two or more calibration components within a set of the devices according to the invention, which have a radiopacity between 0 and 1, have a radiopacity different from each other. In particular embodiments, all of the calibration components in a set have a different radiopacity.

When calibrating the grey values of an area of interest it is of particular interest to have references of grey values which are within the range between the minimal and the maximal grey value presented on the image of the region of interest. In particular embodiments, the resulting grey value of one calibration component in the set is below the average grey value of the region of interest of the body, while the resulting grey value of another calibration component in the set is above the average grey value of the region of interest. Practically, in particular embodiments this implies that the SAR of one calibration component is below the average SAR in the region of interest and the SAR of another calibration component is above the average SAR in the region of interest. In terms of radiopacity, this means that the radiopacity of one calibration component is below the average radiopacity in the region of interest and the radiopacity of another calibration component is above the average radiopacity in the region of interest.

More particularly it is envisaged that for a set of calibration components in the devices of the present invention, the resulting grey value of one calibration component is equal to or below the lowest grey value of the region of interest of the body, while the resulting grey value of another calibration component is equal to or above the highest grey value of the region of interest. Practically, this implies that in particular embodiments, the SAR of one calibration component is equal to or below the lowest SAR in the region of interest and the SAR of another calibration component is equal to or above the highest SAR in the region of interest. In terms of radiopacity, this means that the radiopacity of one calibration component is equal to or below the lowest radiopacity in the region of interest and the radiopacity of another calibration component is equal to or above the highest radiopacity in the region of interest.

The skilled person will understand that the radiopacity and SAR of the calibration components and the region of interest, and the corresponding grey values, depend on the attenuation coefficient of the constituent materials of the calibration components and the region of interest. Specifically, the radiopacity and SAR of a medium depends on the attenuation coefficient of the constituent materials of that medium, and the amount of these materials present in that medium. Thus, the radiopacity and SAR of the calibration components can be varied by varying the size of the calibration components, varying the ratio between the inner and outer diameter of the hollow calibration components, varying the constituent materials of the calibration components, or any combination thereof. In order to identify the optimal materials, size and/or inner and outer diameter for the calibration components, the attenuation coefficients of potential constituent materials of the calibration components can be compared with attenuation coefficients of the bone and tissues expected in the region of interest. Attenuation coefficients of a wide variety of materials, tissues, etc., are available from literature (e.g. J. H. Hubbell and S. M. Seltzer, Tables of X-Ray Mass Attenuation Coefficients and Mass Energy-Absorption Coefficients from 1 keV to 20 MeV for Elements Z=1 to 92 and 48 Additional Substances of Dosimetric Interest, National Institute of Standards and Technology, Gaithersburg, Md.).

The skilled person will further understand that the relation between the attenuation of the radiation used in the medical imaging technique and the attenuation coefficient is given by the Beer-Lambert law.

As explained hereabove, the radiopacity of the calibration components of the devices of the present invention is determined at least in part by the material from which they are made. In particular embodiments, the calibration components are made of one single material. In further particular embodiments, the calibration components are made of a material including a polymer. Polymers often are easily moldable and/or sinterable, which facilitates the production of the calibration components. In order to modify the radiopacity of the calibration components, particles with a different radiopacity may be added to the polymer. Thus, in particular embodiments, the calibration components are made of a polymer containing particles. In further particular embodiments, the particles are made from a material with a higher attenuation coefficient than the polymer, preferably with a higher x-ray attenuation coefficient than the polymer.

Suitable polymers include, but are not limited to, a natural or synthetic rubber or latex, polyvinylchloride, polyethylene, polypropylene, polystyrene, polyamides, polyesters, aramides, polyethyleneterephthalate, polymethylmethacrylate, polymethylmethacrylate or blends thereof. In particular embodiments, the polymer is a polyamide, e.g. nylon. In certain embodiments, the polymer is gutta-percha. The particles may consist of a metal, metal oxide, metal sulfate or any compound containing a metal. Suitable metals include, but are not limited to, barium, iron, lead, titanium, copper, platinum, silver, gold, nickel, zinc or alloys thereof. Additionally or alternatively, particles may consist of iodine or a iodine-containing compound. In particular embodiments, the calibration components and/or the connections between the calibration components consist of a polymer containing Barium sulfate particles. In particular embodiments, the calibration components and/or the connections between the calibration components consist of a blend of nylon (polyamide) with Barium sulfate particles.

Preferably, the connections between the calibration components are not visible in the medical image. Therefore, it is envisaged that in case two or more calibration components are connected via connecting elements, the connecting elements may consist of the same or of a different material than the calibration components. In particular embodiments, the connecting elements may consist of a material which has a lower attenuation coefficient and/or radiopacity than the calibration components. In more particular embodiments, the connecting elements have a radiopacity of zero or close to zero.

For an optimal image correction, the different areas of interest in one image may require different specifications of the calibration components. In particular embodiments, the specifications of the calibration components for certain areas of interest are listed, for example in a table. In further particular embodiments, the calibration components are chosen, designed or manufactured based on a list or table which indicates which calibration devices to use for specific areas of interest, which may include different areas within one image.

For example, as explained herein above, the required radiopacity of the calibration components or the optimal combination of calibration components may depend on the bone and tissues expected in the region of interest. The attenuation coefficients of various tissue types and bones are known, therefore it is possible to design and/or use calibration components for specific regions of interest, or even for specific areas in a region of interest. For example, it is possible to make a table which indicates the type of calibration component (shape, size, material, etc.) which is to be used for a certain region of interest. The table or list may refer to a drawing of a (part of) body, wherein different regions of interest, or different areas within a certain region of interest, are indicated, e.g. numbered.

Thus, in certain embodiments, at least two of the calibration components in a set are adapted to a certain tissue type or a certain region of interest of the body. In further embodiments, all calibration components are adapted to a certain tissue type or a certain region of interest of the body.

The manufacturing of calibration components, and/or the manufacturing of a set of interconnected calibration components which forms a single piece can be ensured in different ways. In certain embodiments, the set of calibration components according to the invention is made by Additive Manufacturing (AM) techniques. Additive Manufacturing (AM) can be defined as a group of techniques used to fabricate a tangible model of an object typically using 3D computer aided design (CAD) data of the object. Currently, a multitude of Additive Manufacturing techniques is available, including stereolithography, Selective Laser Sintering, Fused Deposition Modeling, foil-based techniques, etc.

Selective laser sintering uses a high power laser or another focused heat source to sinter or weld small particles of plastic, metal, or ceramic powders into a mass representing the 3D object to be formed.

Fused deposition modeling and related techniques make use of a temporary transition from a solid material to a liquid state, usually due to heating. The material is driven through an extrusion nozzle in a controlled way and deposited in the required place as described among others in U.S. Pat. No. 5,141,680.

Foil-based techniques fix coats to one another by means of gluing or photo polymerization or other techniques and cut the object from these coats or polymerize the object. Such a technique is described in U.S. Pat. No. 5,192,539.

Typically AM techniques start from a digital representation of the 3D object to be formed. Generally, the digital representation is sliced into a series of cross-sectional layers which can be overlaid to form the object as a whole. The AM apparatus uses this data for building the object on a layer-by-layer basis. The cross-sectional data representing the layer data of the 3D object may be generated using a computer system and computer aided design and manufacturing (CAD/CAM) software.

The dimensions and shape of the calibration devices as produced (e.g. via Additive Manufacturing) may differ slightly from the dimension and shape of the calibration devices as designed. To account for these production tolerances, information about the actual shape and dimensions of the devices may be gathered after production of the devices. In particular embodiments, one or more sets of calibration components of the calibration device described herein are associated with a (digital) identifier after production of the calibration device. The identifier may contain information about the shape and dimensions of the set(s) of calibration components and may be based on a (digital) scan of the device. This ensures that the actual dimensions and shape of the calibration devices can be taken into account for image calibration, which increases the reliability of the calibrated image.

In the calibration devices according to the present invention, the calibration components within a set are fixed or can be provided at different positions, which position(s) ensure that the absorbance of the different calibration components generates optimal information for geometrical and grey-value calibration. This is more particularly achieved by fixed positions which ensure that the lines connecting the center points of the calibration components (e.g. in case of spherical, hemispherical, ellipsoidal, cubic, tetrahedral, pyramidal, disc-shaped and/or washer-shaped calibration components) and or the longitudinal axis of the calibration components (e.g. in case of rod-shaped or wire-shaped calibration components) form a 3D structure.

In particular embodiments, the set of calibration components forms a polyhedron; depending on the nature of the calibration components, this can be achieved in different ways. In particular embodiments, this is ensured by the fact that the center points of at least some of the calibration components form the vertices of an imaginary polyhedron (e.g. in case of spherical, hemispherical, ellipsoidal, cubic, tetrahedral, pyramidal, disc-shaped and/or washer-shaped calibration components). Additionally or alternatively, the longitudinal axis of one or more calibration components may be positioned so as to form the edges of an imaginary polyhedron (e.g. in case of rod-shaped or wire-shaped calibration components). In this context typically only the outer calibration components are taken into account, as the set may comprise one or more calibration components situated at the centre of the imaginary polyhedron. Such an arrangement of the calibration components allows for a stable calculation of the 3D position of the calibration components. In particular embodiments, the set of calibration components forms an imaginary tetrahedron. In particular embodiments, at least one of the calibration components is located inside the imaginary polyhedron. In particular embodiments, one of the calibration components is located in the centre of the imaginary polyhedron. In particular embodiments, the calibration component located inside or in the centre of the imaginary polyhedron, is solid, more preferably a solid sphere. The presence of an inner calibration component facilitates determining the spatial error in the image, and moreover ensures that the set of interconnected calibration components forms a robust and compact structure. In particular embodiments, the calibration component in the center is a large sphere, directly or indirectly connected to other calibration components such as smaller spheres and/or rods.

In particular embodiments, each of the calibration components which form the imaginary polyhedron is directly connected to the calibration component located inside or in the centre of the imaginary polyhedron.

The calibration devices disclosed herein comprising one set of calibration components may be used for a full geometric and grey-value correction of an image. However, different areas in the image may exhibit different distortions. Therefore, an image correction based on the distortions in only one region of the image (i.e. the region wherein the set of calibration components is located), may cause inappropriate corrections in certain other regions of the image. Thus to allow for an improved correction of more regions in the image, it is preferred that the calibration devices according to the present invention comprise more than one set of calibration components. Therefore, in particular embodiments, the medical imaging calibration devices according to the present invention comprise more than one set of interconnected (or interconnectable) calibration components as described above. Some or all of the sets of interconnected calibration components may be identical to each other. In particular embodiments, the calibration device does not comprise identical sets of interconnected calibration components. In certain embodiments, one or each set of interconnected calibration components of the calibration device comprises a unique identifier to distinguish that set from the other sets of the calibration device. For example, a set or one or more of the calibration components thereof may be provided with an identification tag or code which allows identification of the set in the image.

The calibration devices envisaged herein comprise one or more sets of calibration components which calibration components are positioned in specific positions in the device, such that, upon use of the device, the components are provided in the vicinity of the region of interest. More particularly this ensures that the image taken from the region of interest includes the image of the one or more sets of calibration components.

In particular embodiments, the calibration devices according to the present invention comprise, in addition to one or more sets of calibration components, a means for positioning and/or mounting the one or more sets of calibration components on an object of interest such as a body or body part. Typically this feature is not integrated in the calibration components but a separate feature. In particular embodiments, the calibration device comprises more than one feature which can be used for positioning and/or mounting the one or more sets of calibration components on the object of interest.

In particular embodiments, the feature suitable for positioning and/or mounting the set or sets of interconnected calibration components, is a feature which is hereafter also referred to as “means for mounting”, such as a garment. In certain embodiments, the means for mounting is an elastic garment, such as a hose or a sock.

In certain embodiments, at least one set of calibration components of the calibration device described herein is provided with a housing as described above, wherein said housing can be used as a means or feature for positioning. Indeed, the housing may comprise, for instance, an engagement surface suitable for mounting the housing (holding a set of calibration components) on a body or body part. In particular embodiments, the engagement surface is patient-specific, i.e. the engagement surface corresponds to at least a part of a human or animal patient's anatomy. This allows positioning the calibration device on a patient's anatomy in a predefined way and may therefore increase the reliability of the corrected images of the patient's anatomy. The patient-specific engagement surface is typically designed based on a 3D model of a part of the patient's anatomy. The model may be obtained based on 2D or 3D images of the anatomy. In particular embodiments, the housing may comprise one or more free-form structures fitting at least part of the anatomy surface. The term “free-form structure” as used herein refers to a structure having an irregular and/or asymmetrical flowing shape or contour, more particularly fitting at least part of the contour of the anatomy. Thus, in particular embodiments, the free-form structure is a free-form surface. A free-form surface refers to an (essentially) 2D shape contained in a 3D geometric space. Indeed, as will be detailed below, such a surface can be considered as essentially 2D but may have a varying thickness. Typically, the free-form structure or surface is characterized by a lack of rigid radial dimensions, unlike regular surfaces such as planes, cylinders and conic surfaces. Free-form surfaces are known to the skilled person and widely used in engineering design disciplines. Typically non-uniform rational B-spline (NURBS) mathematics is used to describe the surface forms; however, there are other methods such as Gorden surfaces or Coons surfaces. The form of the free-form surfaces are characterized and defined not in terms of polynomial equations, but by their poles, degree, and number of patches (segments with spline curves). Free-form surfaces can also be defined as triangulated surfaces, where triangles are used to approximate the 3D surface. Triangulated surfaces are used in STL (Standard Triangulation Language) files that are known to a person skilled in CAD design. The free-form structures described herein are structured such that they fit the surface of the body parts specifically, thereby giving the structures their free-form characteristics.

Thus, the one or more means for positioning and/or mounting provided in the calibration device described herein is typically provided either separately or comprising one or more of the sets of calibration components attached thereto or housed therein. In particular embodiments, the one or more sets of calibration components can be fixed or are fixed to the means for positioning and/or mounting by a fixation means. The fixation means may be interlocking features, present on the means for mounting and/or the set of calibration components or parts thereof. For example, the calibration components or the connecting elements may contain holes which match with pins on the means for mounting. The sets may also be mounted onto the means for mounting via a snap-fit system. In particular embodiments the calibration device is provided with a means for mounting which is a garment which is designed such that it contains “openings” or “pockets” which can contain the sets of calibration components. Additionally or alternatively the sets of calibration components, or certain calibration components or connections thereof, may be sewn, stapled or glued onto the means for mounting.

In particular embodiments, the one or more sets of calibration components can be provided in predetermined and/or fixed positions on the object of interest. The ability to position the one or more sets of calibration components in a predetermined position by the means for positioning (e.g. engagement surface of the housing) ensures that the calibration components can be positioned on the body in a reproducible way relative to the anatomy of the patient. Additionally or alternatively, the ability to fix the one or more sets of calibration components in one position by the means for mounting ensures that the calibration components can be secured onto the body. Both aspects for positioning and mounting may be ensured by different features but may also be combined in one feature. For example, if the area of interest is located in the foot or leg area of a patient, the means for mounting may be a stocking to which the one or more sets of calibration components are or can be fixed in specific positions, such that upon repeated placement of the sock on the patient, the one or more sets of calibration components are provided in a position relative to the area of interest in the same way every time. The means for positioning and/or mounting may be standard or patient-specific. In particular embodiments, the means for positioning and/or mounting (e.g. such as the housing or the garment) may comprise one or more alignment features which can be used as a visual aid to ensure the repositioning of the means for mounting in the same way on the patient. However, in view of the fact that the presence of the calibration components allows the calibration of the image irrespective of their exact position, implies that the exact reproducibility of the position of the calibration component is not critical. However, it is of interest to ensure a maximal number of calibration components in the vicinity of the area of interest.

In particular embodiments, the means for mounting consists of a material with a low radiopacity, preferably 0 or close to 0. Typical examples of materials envisaged for the means for mounting, particularly garments, include but are not limited to cotton, polyester, nylon, wool, silk, flax and combinations thereof. In particular embodiments, the means for mounting is made of an elastic material such but not limited to a rubber, latex, elasticized cotton, Spandex™, Lycra™, or nylon.

In a further aspect, the use of two or more non-identical geometrical objects having a radiopacity between 0 and 1, for grey-value correction of a medical image is provided. In further embodiments, the use of three or more non-identical geometrical objects having a fixed relative position, at least two of said calibration components having a radiopacity between 0 and 1, for geometric and grey-value correction of a medical image is provided, for example for images obtained via radiography, ultrasound, CT, thermography, MRI or PET. Preferably, the geometrical objects are calibration components as described herein, more particularly the calibration components are part of a calibration device as described herein.

A further aspect provides methods for the acquisition and correction of images of an object such as medical images. More particularly, methods for the correction and/or calibration of images are provided, comprising the steps of:

i) providing an image wherein said image includes, in addition to an image of the object an image of one or more sets of two or more non-identical and interconnected or interconnectable calibration components; and

ii) correcting the image of the object based on the information obtained from the measurement of the geometry and grey values of said image of said calibration components.

In particular embodiments, the object is a body part or a region thereof. In particular embodiments, methods for the acquisition of medical images are provided comprising the steps of:
(I) Application of the non-identical geometrical objects or medical imaging calibration device as described above to the patient in the region of interest, and
(II) Acquisition of a medical image of the region of interest.

The images that are obtained by these methods can be corrected for the spatial positioning of the region of interest, the grey values etc. This allows a more accurate evaluation of the region of interest and further allows a more accurate comparison of images taken at different time points, or even of different patients or different regions of interest. Accordingly, in a further aspect methods are provided for the calibration and/or correction of images such as medical images. Indeed, the methods and tools described herein allow the calibration of medical images, such that particular features on different images can be compared. More particularly, methods for the correction of a medical image to improve its geometrical and/or grey-value accuracy are provided. Based thereon, different images can be compared based on geometrical data (e.g. reflecting size, shape) and/or grey-value (reflecting e.g. density) of features. In particular embodiments, the present invention provides methods for the correction of radiographic images.

In particular embodiments, the methods for the acquisition and/or correction of medical images are non-invasive to the human or animal body.

In particular embodiments, it is of interest to compare different images of the same anatomical feature from the same patient taken at different time points. Additionally or alternatively it is of interest to compare different images of the same anatomical feature in different patients. Further applications will be apparent to the skilled person. The methods according to this aspect of the invention typically comprise a correction of the image. The correction may be performed “live” or “in-line”.

The methods for correction and/or calibration of (medical) images by calibration according to the present invention typically require one or more of the following input data, which can be determined “off-line”:

a) The relative fixed position of the different geometrical objects or calibration components. These are the relative positions of the geometrical objects or calibration components in each set.

b) The relative variable position of the geometrical objects or calibration components. This is the position of the sets of geometrical objects or interconnected calibration components relative to each other and relative to the anatomy of the patient. This relative position is variable, as certain deviations from the average relative position may occur each time an image is acquired, for example due to changes to the anatomy of the patient.

c) The shape parameters of the geometrical objects or calibration components. This includes the shape, inner and outer dimensions, etc. For a geometrical object or calibration component which is a hollow sphere, the parameters comprise the outer diameter, inner diameter, and the information that the geometrical objects or calibration component is spherical.

d) Specific absorption rate of the geometrical objects or calibration components. Alternatively, the attenuation coefficient of the constituent material(s) of the calibration components may be given as input data. The specific absorption rate of the geometrical objects or calibration components can be calculated using the attenuation coefficients and the shape parameters described herein above.

The methods of the present invention involve the application of the geometrical objects or devices of the present invention comprising calibration components in the vicinity of the body part of interest, in a particular position, every time a medical image is taken. While this is not critical, the medical imaging calibration device according to the invention may further facilitate the repeated positioning of the calibration components on the body in the same position.

In particular embodiments, the methods for correction of medical images according to the present invention comprise the steps of:

(I) Providing an image of the region of interest wherein said image includes an image of one or more sets of two or more non-identical geometrical objects or non-identical and interconnected or interconnectable calibration components; and

(II) Correcting the image based on the information obtained from the measurement of the image of the geometrical objects or calibration components.

Thus, the methods of the present invention are based on images obtained from a patient whereby the geometrical objects or calibration device is positioned close to the region of interest. In particular embodiments, step (I) involves providing an image wherein said image includes an image of one or more sets of geometrical objects or calibration components.

In further particular embodiments of the invention, the methods further comprise the acquisition of the images. More particularly the methods comprise the step of:

(I) Application of the medical imaging calibration device as described herein to the patient in the region of interest;

(II) Acquisition of a medical image of the region of interest; and

(III) Correction of the medical image based on the information obtained from the calibration components.

In particular embodiments, methods for the correction and/or calibration of images are provided preferably using the calibration devices as described herein. When making use of the calibration devices such as the devices described herein, images are obtained which can be corrected whenever this is considered necessary.

In particular embodiments of the methods for correction and/or calibration envisaged herein, the correction comprises a grey value correction of the image. Indeed, the image of the calibration components or geometrical objects can be used to correct the grey values of the region of interest. The calibration components or geometrical objects have a known absorption rate and the theoretical grey values can be computed. These can then be compared to the measured grey values to determine the error. The grey-value can then be corrected on the X-ray by applying the error to the X-ray. In particular embodiments this is performed using an error correction table, which provides the errors for different areas in the image.

Additionally or alternatively, the methods for correction of the present invention involve a geometrical correction of the image. Indeed, geometrical accuracy is a common problem in dimensional metrology. Due to various mechanical and optical effects, the image can be distorted. Geometrical correction is done based on the 3D position of the different components or geometrical objects. This can involve determining the 2D positions of each component or object of a set, and, based on the known shape of the component, deducing the 3D positions. However, where the components or objects have known fixed relative positions, and the possible positions can be computed and compared to the image obtained. Comparison of the known relative positions of the components or objects with the measured position, provides the geometrical error. Again, the error can be applied to the image using an error table.

In particular embodiments, the claimed methods involve a combination of a grey-value and geometrical correction of the image.

Additionally or alternatively, the correction of the medical image can comprise an alignment of the patient. For this purpose, different sets of calibration components or geometrical objects are used which are provided on fixed positions in the device. The information on the calibration components or geometrical objects can provide information on the position and/or (part of) the shape of the patient, or a body part of the patient (i.e. the region of interest).

In certain embodiments the methods for correcting and/or calibrating an image comprise the step of:

(I) application of three or more non-identical geometrical objects as described herein to the patient in the region of interest;

(II) acquisition of a medical image of the region of interest; and

(III) grey-value and geometrical correction of the medical image based on the information obtained from the geometrical objects.

In particular embodiments, the methods for correction of medical images calibration envisaged herein comprise one or more or all of the following steps:

(i) Application of the geometrical objects or medical imaging calibration device as described above to the patient at the region of interest. In particular embodiments, the patient wears a garment containing one or more sets of interconnected calibration components or one or more housings comprising the calibration components is positioned on the patient, such that the sets of interconnected calibration components are near the region of interest, and in the field of view of the imaging technique. In further embodiments, the garment or housing(s) contain several sets of calibration components. This allows for a reliable correction in several areas of the image.

(ii) Acquisition of a medical image of the region of interest. Before proceeding with step (ii), step (i) must be fulfilled. Besides the region of interest, also at least one, and preferably all the sets of geometrical objects or interconnected calibration components should be visible on the medical image. More than one image may be acquired during this step.

The methods of the present invention may also be considered to comprise only the correction step, starting from the images obtained as described above in steps (i) and (ii).

(iii) Identification of the geometrical objects or calibration components in the medical image, i.e. the medical image or images obtained in step (ii). Where the geometrical objects or calibration components differ from each other within a set, identification of the different objects or components will be necessary to allow calibration. In particular embodiments, at least two geometrical objects or calibration components are identified for grey-value correction. Similarly, it is envisaged that in particular embodiments, for geometrical correction, at least three geometrical objects or calibration components in one set need are identified. Preferably, all of the geometrical objects or calibration components in one set are identified. Where of interest, all calibration components of all sets are identified. This step can be automated using a computer. For example, this step can involve a search for (images of) hollow spheres, i.e. the geometrical objects or calibration components, on the medical image. Thereby the 2D position of each sphere on the image is determined. Then, each geometrical object or calibration component is identified, for example via the ratio of the outer and inner radius of the spheres. In particular embodiments, the components are tagged or labeled to allow immediate identification in the image.

(iv) Determination of the 3D position of each geometrical object or calibration component. This step is optional. In particular embodiments, the calibration components sets and in each set have fixed known relative positions. For example, each set may consist of four hollow spheres that form a tetrahedron with a hollow or solid sphere in the centre of the tetrahedron. Then, each possible position of a tetrahedron corresponds to a different projected silhouette on the medical image, thus each possible position of the calibration components can be identified from the image.

As detailed above, depending on the nature of the correction envisaged, the methods may comprise one or more of the following steps:

1. Calculation of the geometrical error on the medical image. The geometrical error can be determined from the information obtained in step (iv), the position of the central calibration component in each set and the 2D position of the central calibration component on the medical image. A more accurate calculation of the geometrical error is obtained by using multiple sets of calibration components.

2. Geometrical correction of the medical image. In particular embodiments, an error table is constructed is based on the geometrical error calculated in step (v), and this error table is applied to the image.

3. Correction of the geometrical object or calibration component set positions. In particular embodiments, the positions of the set or sets of calibration components are corrected based on the error table constructed in step 2.

4. Determination of the position and some shape parameters of the patient. The positions of the geometrical objects or calibration components are known from the previous steps. The means for mounting the set or sets of geometrical objects or calibration components on the body is designed in such a way that it always positions the set or sets of calibration components in the same way relative to the anatomy of the patient. Then the position of the patient can be obtained from the medical image. Although the relative positions of the calibration components can deviate to some extent, the deviations average out upon optimization of the registration. The deviation from the average then also gives basic information about the anatomy and thus information about some shape parameters of the patient.

5. Calculation of the grey-value errors in the image. The specific absorption rate of the geometrical objects or calibration components is known and part of the input, thus for all the geometrical objects or calibration components that are not obstructed by the anatomy of the patient, the theoretical grey values can be determined. The differences with the measured grey-values are then the errors.

6. Correction of the grey-values on the medical image. In particular embodiments, an error table is constructed is based on the grey-value error calculated in step 5, and this error table is applied to the image. Preferably, this error table is a set of local grey-value error tables which can be spatially interpolated. Different local grey-value error tables can be obtained by placing a set of calibration components in different areas in the image.

In particular embodiments, the methods for correction of medical images envisaged herein further comprise the step of removing the geometrical objects or medical imaging calibration components from the medical image. Since the position of the calibration components is known and also their effect on the medial image is known via their known specific absorption rate, the effect of the calibration components can be removed from the medical image. This results in a medical image which shows no artifacts arising from calibration components anymore.

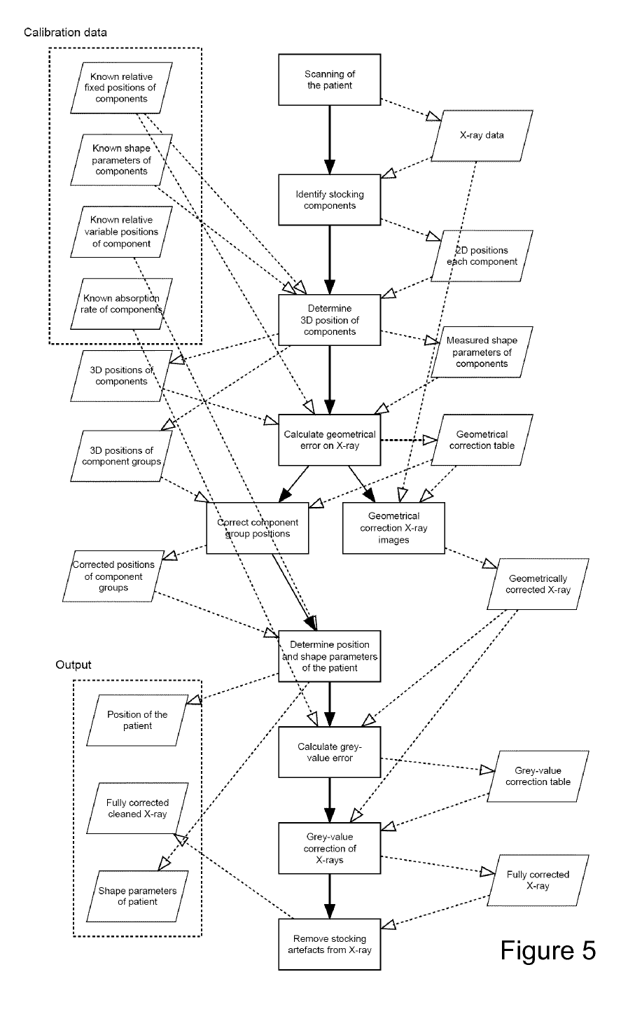

FIG. 5 shows a flow diagram of exemplary steps in a method for correction of medical images according to the present invention. The diagram shows possible steps of the method (rectangles and arrows with full line), together with the input and output for every step (parallelograms and arrows with dotted line).

The calibration devices and methods according to the present invention can be used for calibration of images obtained by x-ray radiography, ultrasonography, MRI or CT. Accordingly, a further aspect of the present invention provides the use of the medical imaging calibration devices as described above, for quantitative measurement on data obtained from x-ray radiography, ultrasonography, MRI or CT.

In a further aspect computer program products for performing geometrical and/or grey-value correction on images obtained by medical imaging, as described above, are provided comprising:
a computer readable medium; and
software instructions, on the computer readable medium, for enabling the computer to perform some or all of the following operations:
   Identification of the calibration components (or geometrical objects in a medical image;
   Calculation of the geometrical error on the medical image based on the image of the calibration components (or geometrical objects) and geometrical correction of the medical image;
   Correction of the calibration component (or geometrical objects) set positions;
   Determination of the position and shape parameters of the patient, based on the image of the calibration components (or geometrical objects);
   Calculating of the grey-value error on the medical image based on the image of the calibration components (or geometrical objects) and correcting the grey-values in the medical image;
   Removing the medical imaging calibration artifacts from the medical image.
      The present invention will be illustrated by the following non-limiting embodiments.

### EXAMPLES

### Example 1

### Development and Use of the Calibration Device

### a) Development of the Calibration Device

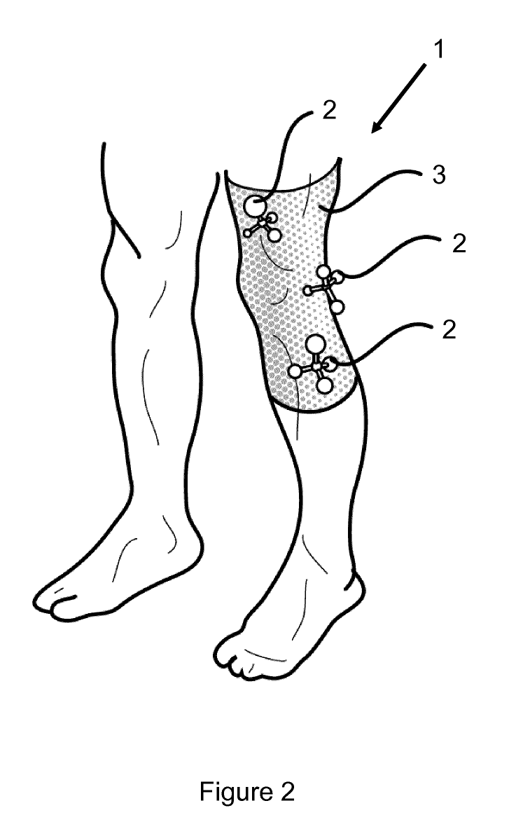

The calibration device (1) comprises several sets (2) of calibration components, as shown in FIG. 2.

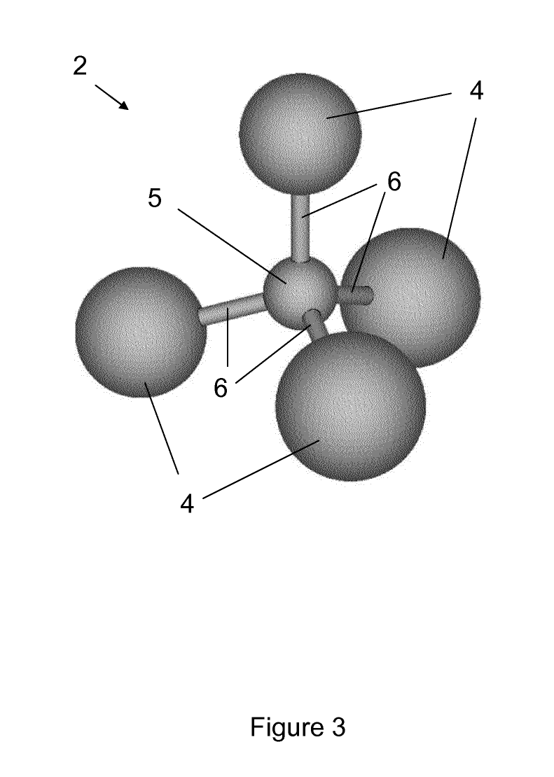

An example of a set (2) according to a particular embodiment of the present invention is shown in FIG. 3. The set (2) consists of five calibration components (4, 5), which may be solid spheres with varying diameters, and/or hollow spheres with varying ratios between the large and the small radius (not shown). The varying diameters and/or ratios facilitate identification of the spheres. Moreover, the difference in overall diameter and/or thickness of the shell ensures differences in absorption rate between the spheres. The outer calibration components (4) are interconnected such that the lines connecting their centers form the edges of a tetrahedron, with one calibration component (5) in the center. Thus, there are four outer calibration components (4) and one inner calibration component (5). All outer calibration components are directly connected to the inner calibration component via connections (6).

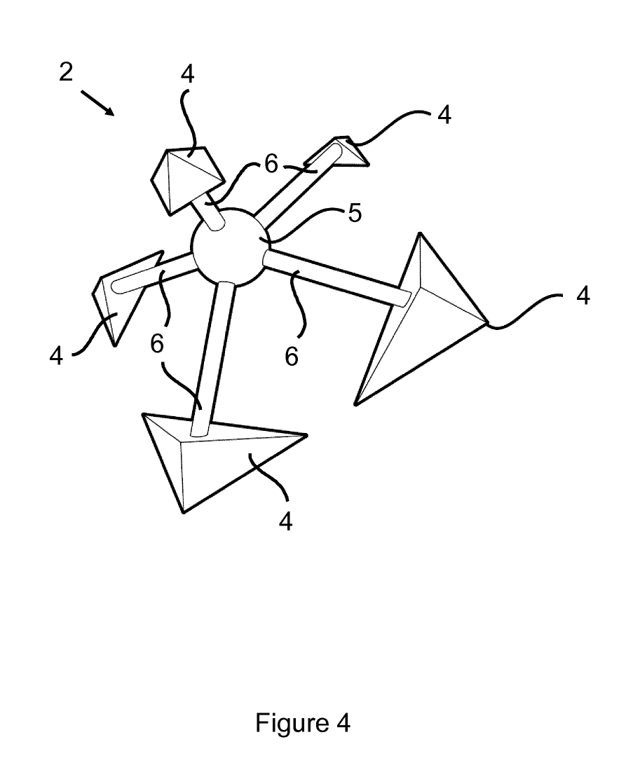

An example of a set (2) according to another embodiment of the present invention is shown in FIG. 4. The set (2) consists of six calibration components (4, 5), which may be solid or hollow spheres, tetrahedrons or pyramids. The tetrahedrons and pyramids may be regular or irregular. The outer calibration components (4) are interconnected such that the lines connecting their centers form the edges of a pyramid, with one calibration component (5) in the center. Thus, there are five outer calibration components (4) and one inner calibration component (5). All outer calibration components are directly connected to the inner calibration component via connections (6).

As shown in FIG. 2, the calibration device (1) further comprises a means (3) for mounting the set(s) of calibration components. The means (3) for mounting is a garment which is designed in such a way that it positions the sets of calibration components always in the same way relative to the anatomy of the patient. Furthermore, the sets of calibration components are located near to or on the region of interest, and on different places on the stocking, in such way that different regions in the image each contain one or more sets of calibration components.

### b) Application of the Device and Scanning of the Patient

The stocking comprising the sets of calibration components is applied on the patient in a specific position and one or more x-ray images are taken.

### c) Identifying the Stocking Components

A computer program is used to identify the hollow spheres (based on the ratio of inner/outer radius) and determine the position of the spheres in the 2D-image.

### d) Determining the 3D Position of Each Component

It is theoretically possible to determine the 3D position of a sphere based on the 2D position and shape parameters of its projected silhouette. The projected silhouette of a hollow sphere is 2 “concentric” ellipses. The eccentricity of the two ellipses indicates the (projective) distance from the perpendicular projection axis. The ratios between the measured (small) radii and the known 3D radii gives the Z-position of the sphere (the scale factor).

However, a metrologically more stable solution is used as the spheres in the sets have a fixed known relative position, forming a tetrahedron and a hollow sphere in the centre of the tetrahedron. Indeed, each possible position of a tetrahedron corresponds to a different projected silhouette and can therefore be identified from the X-ray.

### e) Calculating the Geometrical Error on the X-Ray

Having used the 4 outer spheres to identify the 3D position of each set of calibration components and knowing the nominal position of the centre sphere, we compare it with its measured position, providing us with the local error. As the calibration device comprises more than one set of calibration components, more than one local error can be calculated. Then, the local errors can be spatially interpolated.

### f) Geometrical Correction of the X-Ray Images

This is standard procedure. An error table is made and this is simply applied to the X-ray. The positions of the sets of calibration components are corrected based on the error table.

### g) Determining the Position and (Part of) the Shape Parameters of the Patient

The stocking can now also be used to determine the alignment of the patient. We have calculated the different positions of the sphere groups. These groups are attached to the stocking. As the stocking is designed in such a way that it positions the sphere groups always in the same way relative to the anatomy of the patient, deviations from the position of the spheres are indicative of changes in the anatomy of the patient. There will be deviations obviously because the patient is different. But if an optimization on the registration is used, these differences are averaged out. The variation to the average gives some basic information about the anatomy of the patient. This information is also part of the output.

### h) Calculation of the Grey-Value Error and Grey-Value Correction of the X-Ray

The hollow spheres are now used to calculate the grey-value error. We know what the absorption rate should be and it can be measured. For all the spheres that are not obstructed by the patient, the theoretical grey values can be computed. The differences with the measured grey-values are the errors. The grey-value correction of the X-ray is performed by applying an error correction table. This error correction table is a set of local grey-value error tables which can be spatially interpolated.

### Example 2

### Particular Embodiments of Interconnected Calibration Components

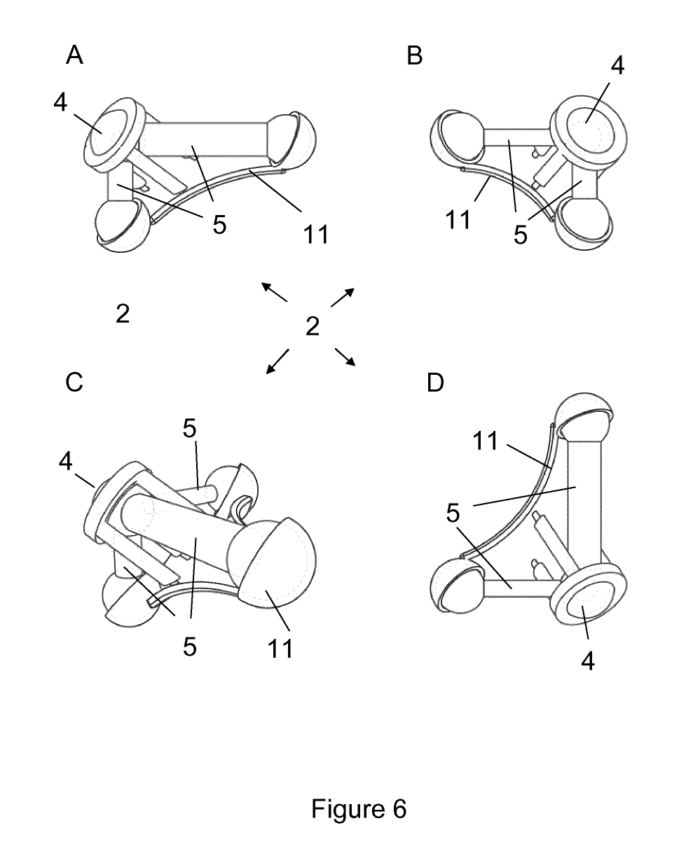

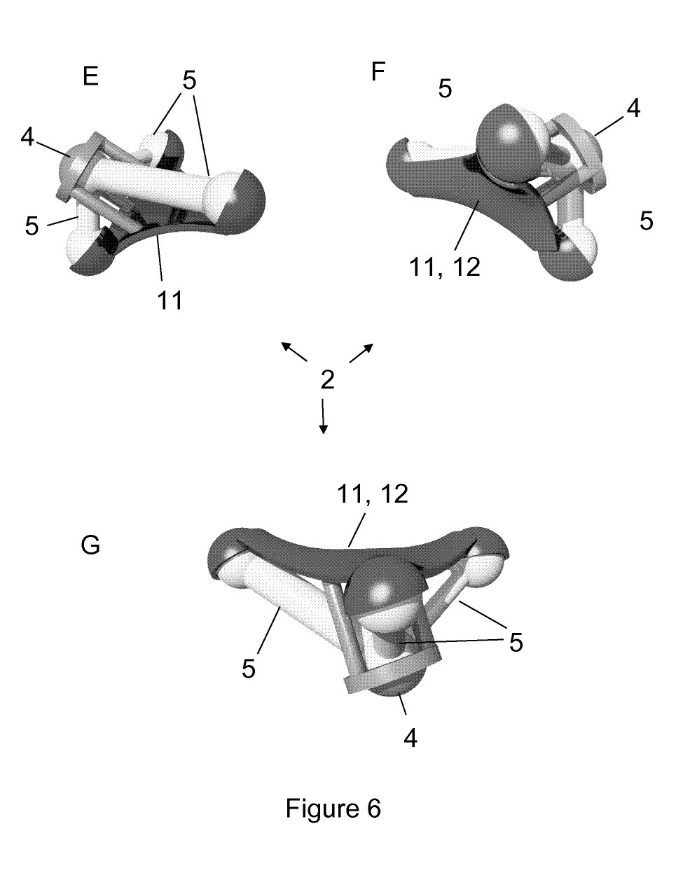

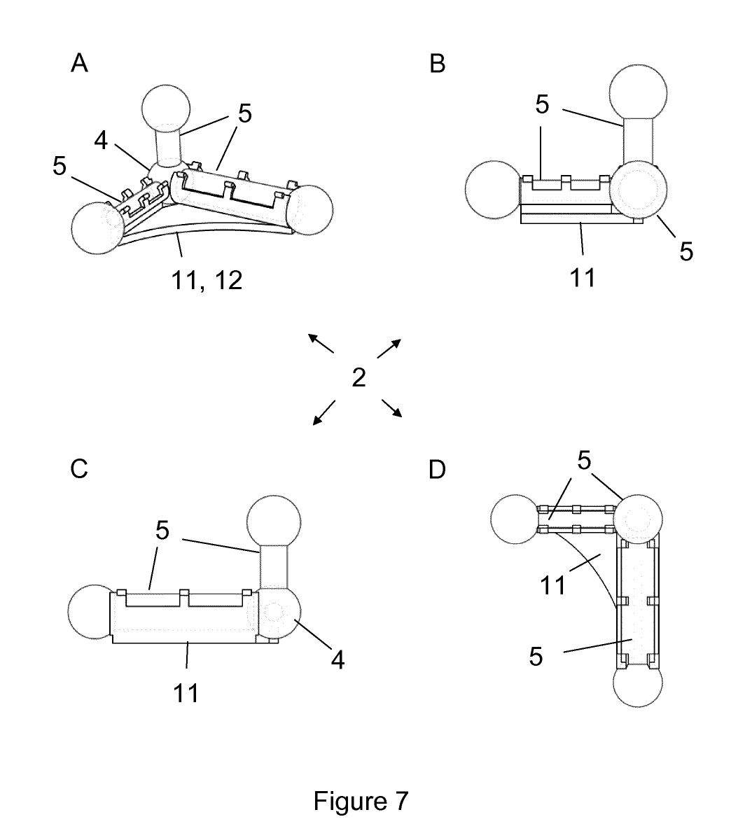

FIG. 6 (A-G) shows a set (2) of interconnected calibration components (4, 5) according to a particular embodiment of the present invention. The set comprises one spherical calibration component (4) connected to three calibration components (4′), each consisting of a solid cylindrical rod and a sphere. The rods form the edges of a tetrahedron, whereas the spheres form the vertices of the tetrahedron. The combination of the spheres and rods allows for a full 3D geometric and grey-value correction of a medical image such as a radiographic image. The spheres are identical, but can be identified in an x-ray image because each of the rods of the calibration components has a unique width.

The relative position of the spheres is fixed via the rods and further secured via a housing (11) holding the spheres. The housing also encapsulates the spheres, thereby protecting their structural integrity. The housing consists of two removably detachable parts, such that the set (2) can easily be inserted into the housing and removed therefrom. The housing is made of a material with a low radiopacity, such as a polymer. The housing further comprises a patient-specific engagement surface (12) for positioning the housing on a patient's body part. This ensures an accurate positioning of the calibration components on the body part.

FIG. 7 (A-D) shows a similar set (2) of interconnected calibration components (4,5) as shown in FIG. 6, in a different housing (11). The housing (11) has a triangular shape and clips on the two longest rods of the calibration components, thereby securing their relative positions. The stiffness and strength of the shortest rod is sufficient as such and does not require further support from the housing. The housing further comprises an engagement surface (12) for positioning the housing on a patient's body part.

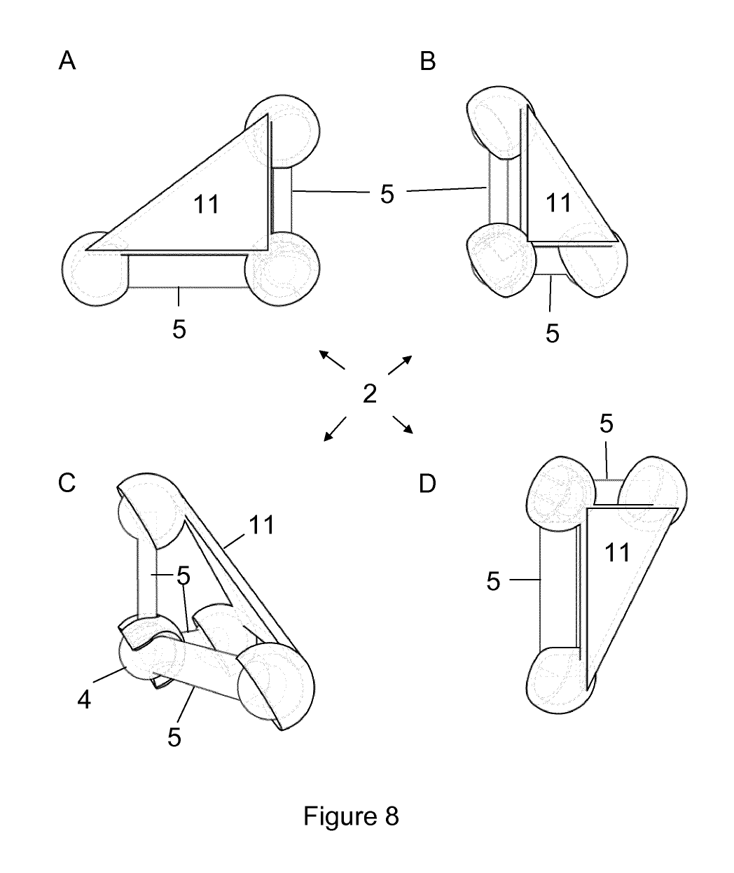

FIG. 8 (A-D) shows a similar set (2) of interconnected calibration components (4,5) as shown in FIG. 6, in a different housing (11). The housing (11) comprises four hollow hemispheres and is designed such that it clips on one of the spheres of the calibration device while holding the three others, thereby securing their relative positions.

### Example 3

### Particular Embodiments of Interconnected Calibration Components

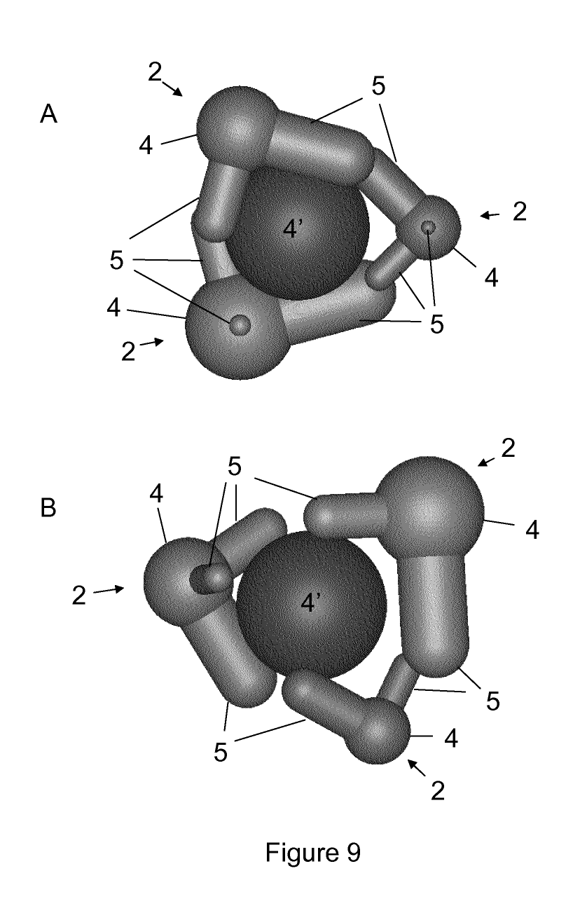

FIG. 9 A and B illustrate a configuration of three sets (2) of interconnected calibration components (4, 5) according to a particular embodiment of the present invention. Each set (2) comprises one spherical calibration component (4) and three rod-like calibration components (5). The rods form edges of a tetrahedron, whereas the sphere forms a vertex of the tetrahedron.

The spherical calibration components from the three sets have a different diameter, which facilitates identification of the sets. Furthermore, the different diameter of the spheres results in a different radiopacity for each sphere.

The rod-shaped calibration components of each set are different from each other. More particularly, the rods have a different diameter and/or length. This facilitates identification of the rods, and results in a variety of radiopacities. Each rod in a set is positioned perpendicularly to the other rods of that set. This facilitates image correction.

For further facilitation of the image correction, the sets (2) may be arranged such that two or more rods of the different sets are parallel to each other. The sets may further be arranged in a specific way relative to one or more calibration components (4′) which may or may not belong to a set of interconnected calibration components.

### Example 4

### Particular Embodiments of Interconnected Calibration Components

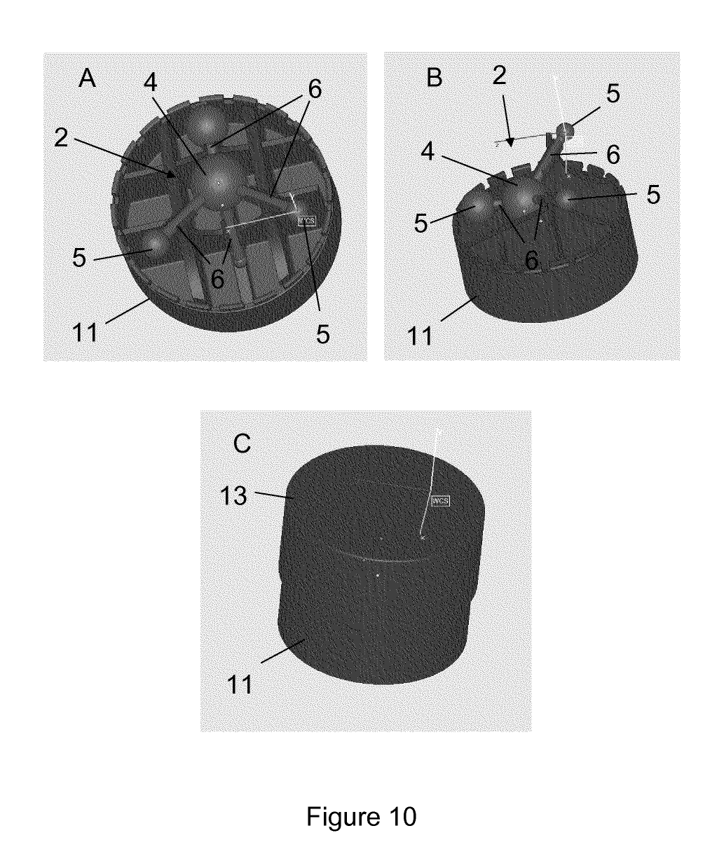

FIG. 10 (A-C) shows a set (2) of interconnected calibration components (4, 5) according to a particular embodiment of the present invention. The set comprises five spherical calibration components (4, 5). The centers of the outer spherical calibration components (5) form the vertices of a tetrahedron. The outer calibration components (5) are connected to the inner spherical calibration component (4) via rod-like connecting elements (6). The rods (6) between the calibration components may also be used as calibration components.

The spherical calibration components (4, 5) have different diameters and different radiopacities. Thus, the combination of the spheres (and optionally the rods) allows for a full 3D geometric and grey-value correction of a medical image such as a radiographic image.

The relative position of the spheres is fixed via the rods and further secured via a housing (11) which clips on the rods (6). The housing may protect the set of interconnected calibration components during transport. Additionally or alternatively, the housing may be made of a radiolucent material and can be used protect the set during acquisition of a medical image. The housing may comprises a lid (13) for further protection of the set (2).

1. A method for correction of an image, comprising the steps of:
i) providing an image wherein said image includes an image of one or more sets of two or more non-identical and interconnected calibration components configured to form a three-dimensional geometrical object, wherein said calibration components differ at least in radiopacity; and
ii) correcting the geometry and grey values of said image, based on the information obtained from the measurement of the geometry and grey values of said image of said calibration components.

2. The method according to claim 1, which comprises:
identification of two or more calibration components in the image;
determination of the location and the grey value of said two or more components in the image;
calculating the grey value error based on the difference between the determined grey value and the calculated grey value; and
applying said correction on said image.

3. The method for correction of images according to claim 1, wherein step i) comprises providing an image wherein said image includes an image of one or more sets of three or more non-identical calibration components, wherein said two or more non-identical calibration components differ at least in radiopacity and are interconnected to form a fixed three-dimensional geometrical figure.

4. The method according to any one of claim 1, which comprises
identification of at least three calibration components in the image;
determination of the three-dimensional position of each calibration component;
calculating of the geometrical error on the image; and
applying said correction on said image.

5. The method according to claim 1, wherein said image comprises an image of at least part of a patient and said correction comprises providing information on the position and the shape of said part of said patient.

6. The method according to claim 1, wherein said correction is further based on the determination of errors in more than one area in the image.

7. The method according to claim 1, wherein said correction is an in-line correction.

8. A calibration device for imaging, such as for medical imaging, comprising two or more non-identical and interconnected calibration components, wherein at least two of said calibration components have a different radiopacity which is between, and not including, 0 and 1, and wherein said calibration components are configured to form a fixed three-dimensional geometrical object.

9. (canceled)

10. The calibration device according to claim 8, which further comprises a separate feature for positioning and/or mounting said calibration components on the body.

11. (canceled)

12. The calibration device according to claim 8, wherein said calibration components comprise one or more elements selected from spheres and rods.

13. The calibration device according to claim 8, wherein the centers or longitudinal axes of said interconnected calibration components respectively form the vertices or edges of an imaginary polyhedron.

14. The calibration device according to claim 13, wherein said interconnected calibration components forms a tetrahedron.

15. The calibration device according to claim 13, wherein said interconnected calibration components forms a polyhedron with at least one calibration component placed inside the polyhedron.

16. (canceled)

17. The calibration device according to claim 8, further comprising a housing for holding two or more of said calibration components.

18. The calibration device according to claim 17, wherein said housing comprises a patient-specific engagement surface for positioning said housing on the body.

19. The calibration device according to claim 8, where the outer diameter of at least one of said calibration components is different from the outer diameter of another calibration component.

20. The calibration device according to claim 8, containing at least two sets of interconnected calibration components.

21. The calibration device according to claim 10, wherein said dedicated feature for positioning and/or mounting said calibration components is a garment.

22. (canceled)

23. A calibration device comprising a set of at least five non-identical spheres which have an x-ray radiopacity between and not including 0 and 1, wherein said spheres are interconnected or interconnectable in such way that the centers of at least four of said spheres form the vertices of an imaginary polyhedron, and one of said spheres is located inside said polyhedron.

24-26. (canceled)
